(19) United States
(12) Patent Application Publication
(10) Pub. No.: US 2024/0000989 A1
(43) Pub. Date: Jan. 4, 2024

(54) MALODER NEUTRALIZING COMPOSITION

(71) Applicants: Innovasource, LLC, Huntersville, NC (US); Intarome Fragrance Corporation, Norwood, NJ (US)

(72) Inventors: Glenn F. Cueman, Denver, NC (US); Andrew F. Fay, Granite Falls, NC (US); Kenneth Edward McAlister, Orem, UT (US); Sandra N. Porter, Huntersville, NC (US)

(73) Assignees: Innovasource, LLC, Huntersville, NC (US); Intarome Fragrance Corporation, Norwood, NJ (US)

(21) Appl. No.: 17/827,254

(22) Filed: May 27, 2022

(51) Int. Cl. A61L 9/01; A61L 2/16

(52) U.S. Cl. CPC A61L 9/01; A61L 2101/36; A61L 2/16

(57) ABSTRACT

A malodor neutralizing composition containing derivatives of undecylenic acid, wherein the composition includes a ratio of ethyl ester to methyl ester from 5/95 to 30/70 that comprises from about 20 wt. % to about 90 wt. % of the composition and heptyl ester comprises from about 10 wt. % to about 80 wt. % of the composition, wherein the sum of the wt. % of the composition is 100%.

### FIELD OF THE INVENTION

The present invention relates generally to a malodor neutralizing composition, and more generally relates to a low intrinsic odor malodor neutralizing composition containing undecylenic acid derivatives.

### BACKGROUND OF THE INVENTION

A malodor has a very unpleasant or offensive smell, odor, or stench. When a malodor exists in a habitable environment, such as a residence or workplace, or a confined space, such as a vehicle, it disrupts the enjoyment of everyone within these environments or spaces. Malodor may be caused by mold, waste, smoke, feces and urine. Additionally, malodor may be kitchen odors, caused by the preparation or use of onions, fish, curry, garlic, and the like. There are many examples of malodors and the specific malodors listed are not exhaustive, but an exemplary list of common malodors commonly encountered by individuals.

A perfume or fragrance may be utilized to counteract the malodor, but it is difficult for a perfume or fragrance by itself to mask a malodor. These perfumes or fragrances attempt to cover up or mask the malodor, but are generally ineffective in masking all types of malodors. A malodor neutralizing composition needs to be utilized to neutralize a wide variety of malodors. A malodor neutralizing composition does not mask a malodor, like a perfume or fragrance, instead, it counteracts the malodor, reducing the intensity, by neutralizing the malodor.

Prior art solutions for neutralizing malodors, include U.S. Pat. No. 6,495,097 that discloses combining methyl ester and ethyl ester derivatives of undecylenic acid with a fragrance or flavor. U.S. Pat. No. 8,741,275 discloses the use of undecylenic acid and citric acid with an ester, such as benzyl acetate, ethyl heptanate, and methyl benzoate. None of the prior art discloses a composition as disclosed herein that is effective at neutralizing malodors and with a low intrinsic odor.

### BRIEF SUMMARY OF THE INVENTION

According to an embodiment of the present invention, a malodor neutralizing composition includes an undecylenic acid or a derivative thereof, wherein the composition comprises derivatives of undecylenic acid of about 1 wt. % to about 99 wt. % methyl ester, about 1 wt. % to about 99 wt. % ethyl ester, and about 1 wt. % to about 99 wt. % of a derivative of undecylenic acid with a molecular weight of between about 250 and 300, wherein the sum of the wt. % of the composition is 100%.

According to another embodiment of the present invention, the malodor neutralizing composition includes an undecylenic acid or a derivative thereof, wherein the composition comprises derivatives of undecylenic acid of about 1 wt. % to about 99 wt. % methyl ester, about 1 wt. % to about 99 wt. % ethyl ester, and about 1 wt. % to about 99 wt. % heptyl ester, wherein the sum of the wt. % of the composition is 100%.

According to yet another embodiment of the present invention, the malodor neutralizing composition includes about 10 wt. % to about 80 wt. % heptyl ester.

According to yet another embodiment of the present invention, the malodor neutralizing composition includes about 20 wt. % to about 80 wt. % methyl ester.

According to yet another embodiment of the present invention, the malodor neutralizing composition includes about 20 wt. % to about 80 wt. % ethyl ester.

According to yet another embodiment of the present invention, the malodor neutralizing composition includes a ratio of ethyl ester to methyl ester from 5/95 to 30/70 that comprises from about 20 wt. % to about 90 wt. % of the composition and the heptyl ester comprises from about 10 wt. % to about 80 wt. % of the composition, wherein the sum of the wt. % of the composition is 100%.

According to yet another embodiment of the present invention, the malodor neutralizing composition includes a ratio of ethyl ester to methyl ester of 80/20 that comprises about 70 wt. % of the composition and the heptyl ester comprises about 30 wt. % of the composition, wherein the sum of the wt. % of the composition is 100%.

According to yet another embodiment of the present invention, a composition for reducing malodors includes a component selected from the group consisting of fragrances, flavors, and mixtures thereof, and an undecylenic acid or a derivative thereof, wherein the composition comprises a derivative of undecylenic acid of about 1 wt. % to about 99 wt. % methyl ester, about 1 wt. % to about 99 wt. % ethyl ester, and about 1 wt. % to about 99 wt. % heptyl ester, wherein the sum of the wt. % of the composition is 100%.

### BRIEF DESCRIPTION OF THE DRAWINGS

The present invention is illustrated and described herein with reference to the various drawings, in which like reference numbers denote like method steps and/or system components, respectively, and in which:

FIG. 1 graphically shows the percent of ethyl ester and methyl ester in the malodor neutralizing composition compared to the prior art.

### DETAILED DESCRIPTION OF THE INVENTION

The present invention may be understood more readily by reference to the following detailed description of the invention taken in connection with the accompanying drawing FIGURES, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The ranges set forth herein include both numbers at the end of each range and any conceivable number there between, as that is the very definition of a range. It is therefore to be understood that the ranges and limits mentioned herein include all ranges located within the prescribed limits (i.e., subranges). For instance, a range from about 100 to about 200 also includes ranges from 110 to 150, 170 to 190, 153 to 162, and 145.3 to 149.6. Further, a limit of up to about 7 also includes a limit of up to about 5, up to 3, and up to about 4.5, as well as ranges within the limit, such as from about 1 to about 5, and from about 3.2 to about 6.5 as examples.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

As used herein, the terms “comprise”, “comprises”, “containing”; and “has”, “have”, “having”; and “includes”, “include” and “including”; are open-ended transition terms used to transition from a subject recited before the term to one or more elements recited after the term, where the element or elements listed after the transition term are not necessarily the only elements that make up the subject.

The term weight percent or wt. % means the weight of a given a material relative to the weight of the resulting composition which includes the raw material. For example, a composition having 30 wt. % of heptyl ester means that the composition includes 30 parts by weight of the heptyl ester material relative to 100 parts of the total weight of the resulting composition.

The present invention is directed to a malodor neutralizing composition that comprises an undecylenic acid or derivatives thereof. The malodor neutralizing composition is effective in malodor control and has a lower intrinsic odor. It has been discovered the combination of undecylenic acid or derivatives thereof with an ester in the molecular weight range of between 250 and 300 provides a composition that is effective in malodor control and has a lower intrinsic odor.

The term “undecylenic acid” herein is commonly referred to as undec-10-enoic acid (CH₂═CH(CH₂)₈COOH). Undecylenic acid is any of several isomeric straight-chain unsaturated acids C₁₁H₂₀O₂ that is a naturally occurring acid found in tears and sweat. Undecylenic acid is an organic unsaturated fatty acid commonly derived by cracking C₁₈, often derived from castor oil, into C₇ and C₁₁ (where C₁₁ is known a undecylenic acid or undecanoic acid) under pressure. Undecylenic acid is used in the manufacture of pharmaceuticals, cosmetics, perfumes, anti-dandruff shampoos, and anti-microbial powders. Undecylenic acid is a natural fungicide and is FDA approved in over-the-counter medications for skin disorders or problems.

A malodor neutralizing composition refers to a composition with properties for neutralizing malodors. The malodor neutralizing composition may be added or mixed with other components, such as a perfume, fragrance, flavor, wax, and the like.

Ester derivatives of undecylenic acid may be utilized in accordance with the present invention that are water-soluble as well as lipid-soluble. The ester derivatives of undecylenic acid utilized in the present invention are methyl ester (C₁₂H₂₂O₄), ethyl ester (C₁₃H₂₄O₂) and heptyl ester (C₁₈H₃₄O₂). The composition comprises the derivatives of the undecylenic acid from about 1 wt. % to about 99 wt. % methyl ester, from about 1 wt. % to about 99 wt. % ethyl ester, and from about 1 wt. % to about 99 wt. % heptyl ester, wherein the sum of the wt. % of the composition is 100%. Preferably, the composition comprises the derivatives of the undecylenic acid from about 20 wt. % to about 80 wt. % methyl ester, from about 20 wt. % to about 80 wt. % ethyl ester, and from about 10 wt. % to about 80 wt. % heptyl ester, wherein the sum of the wt. % of the composition is 100%. The malodor neutralizing composition can be combined with fragrance and/or flavor compositions for inclusion in a variety of consumer products.

Table A lists the preferred ranges of the malodor neutralizing composition of the present invention;

TABLE A

| Undecylenic derivative | Acceptable Range | Preferred Range |
|---|---|---|
| ester | (wt. %) | (wt. %) |
| Methyl Ester | 1-99 | 20-80 |
| Ethyl Ester | 1-99 | 20-80 |
| Heptyl Ester | 1-99 | 10-80 |

The ratio of ethyl ester to methyl ester in the composition may be between 2/1 to 5/1, wherein the composition comprises between 20 wt. % to 90 wt. % of a combination of ethyl ester and methyl ester. The remaining portion of the composition comprises heptyl ester in the range from about 10 wt. % to about 80 wt. %, wherein the sum of the wt. % of the composition is 100%. More preferably, the ratio of methyl ester in the composition may be between 3/1 to 5/1, wherein the composition comprises between 20 wt. % to 90 wt. % of a combination of ethyl ester and methyl ester. The remaining portion of the composition comprises heptyl ester in the range from about 10 wt. % to about 80 wt. %, wherein the sum of the wt. % of the composition is 100%. Most preferably, the ratio of ethyl ester to methyl ester in the composition is about 4/1, wherein the composition comprises about 70 wt. % of a combination of ethyl ester and methyl ester. The remaining portion of the composition comprises heptyl ester in the range of about 30 wt. %, wherein the sum of the wt. % of the composition is 100%.

The malodor neutralizing composition of the present invention may be combined with a wide variety of fragrancing ingredients, such as a perfume, a fragrance, a flavor, or mixture thereof. The resultant composition or mixture may be referred to as a “pre-mix” or “ONP.” The pre-mix, containing the malodor neutralizing composition, may be absorbed onto a carrier, ensuring the fine distribution of the malodor neutralizing composition and controlled release during use. The carrier may be fragranced or unfragranced. Such carriers may include light sulfates, salts, silica gels, borates, carbonates, soda ash, tripolyphosphates, zeolites, gypsums, clays, granulated clays, slurries, starches, polyethylene, microporous high density polyethylene (HDPE), and cellulose-based materials.

The pre-mix, containing the malodor neutralizing composition, may also be microencapsulated or in spray-dried form or in the form of inclusion complexes or extrusion products.

The malodor neutralizing composition may also be incorporated into the wax of a candle, allowing the ester derivatives of undecylenic acid (methyl ester, ethyl ester, and heptyl ester) to be released into the surrounding air while the candle burns. The incorporation into a candle allows the malodor neutralizing composition to be dispersed within the surrounding air without the need for spraying by a mechanical device.

The malodor neutralizing composition may be incorporated into many household and personal care products. By way of example only, the household products include laundry detergent, air fresheners, bleach, fabric softener, disinfection products, and cat litter. The personal care products, include, but are not limited to, shampoo, conditioner, mousse, gel, both gel, deodorant, personal hygiene products, chewing gum, mouthwashes, powder, and cream.

The malodor neutralizing composition is prepared by mixing the undecylenic esters together mechanically to form a homogenous composition. Preferably, the malodor neutralizing composition as described herein is prepared by mixing the ethyl ester, methyl ester, and heptyl ester together mechanically to form a homogenous composition. More preferably, the malodor neutralizing composition as described herein is prepared by initially mixing the ethyl ester and methyl ester together mechanically to form a homogenous mixture with a preferred ratio of ethyl ester to methyl ester. The mixture of ethyl ester to methyl ester is combined with the heptyl ester by mixing the mixture containing ethyl ester and methyl ester with heptyl ester to form a homogenous composition.

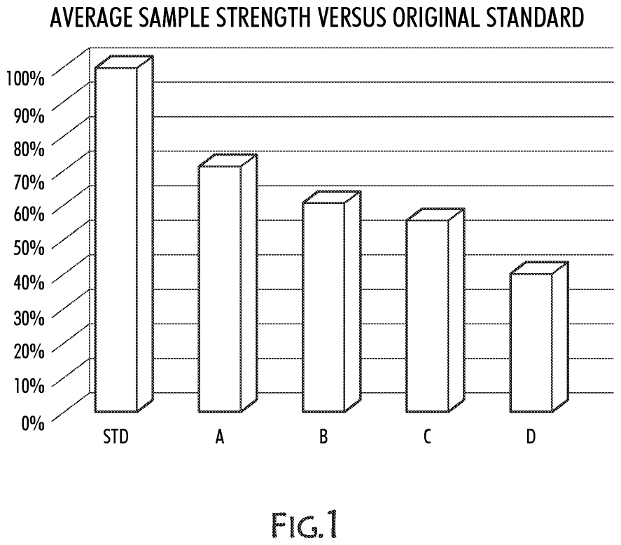

A prior art composition containing only ethyl ester and methyl ester and disclosed in U.S. Pat. No. 6,495,097 is comprised of a ratio of ethyl ester to methyl ester of 4/1. FIG. 1 illustrates the strengths of the samples of the present invention as compared to the Prior Art composition, containing a ratio of ethyl ester to methyl ester of 4/1 and referred to herein as “Prior Art” or “Prior Art composition.” Table B contains the numeric values represented in FIG. 1.

TABLE B

| Sample | Prior Art | A | B | C | D |
|---|---|---|---|---|---|
| Avg. Score | 100% | 71.4% | 60.7% | 55.7% | 40.4% |

The composition of the samples are set forth in Table C below:

| Sample | Ethyl ester/methyl ester (wt. %) | Heptyl ester (wt. %) |
|---|---|---|
| Prior Art | 100 | 0 |
| A | 90 | 10 |
| B | 70 | 30 |
| C | 50 | 50 |
| D | 20 | 80 |

### Example

An evaluation panel of 25 participants was assembled and requested to evaluate the intrinsic odor of the malodor neutralizing composition as compared to the Prior Art composition. The participants were provided a sample of the Prior Art composition and samples of the malodor neutralizing composition set forth in Table D. Compositions for samples A, B, C, and D were prepared in accordance with the present invention by mixing the undecylenic esters together to form a homogenous composition.

Compositions for each of the samples A, B, C, and D and the Prior Art were each placed in separate bottles. Each bottle was affixed with a pump sprayer for dispensing the composition contained within the bottles. The compositions for samples A, B, C, and D and the Prior Art were sprayed separately on aerosol testing blotters with the respective blotter labeled with a designation indicating the specific composition sprayed on the particular aerosol testing blotter. In this example, the aerosol testing blotters were labeled A, B, C, and D to depict the specific sample composition sprayed on the aerosol testing blotter. Another aerosol testing blotter was labeled STD to refer to the Prior Art composition sprayed on this particular aerosol testing blotter.

TABLE D

| Sample | Ethyl ester/methyl ester (wt. %) | Heptyl ester (wt. %) |
|---|---|---|
| Prior Art | 100 | 0 |
| A | 90 | 10 |
| B | 70 | 30 |
| C | 50 | 50 |
| D | 20 | 80 |

Each of the 25 participants of the evaluation panel were asked to sniff each aerosol testing blotter containing compositions of samples A, B, C, and D and compare to an aerosol testing blotter containing the Prior Art composition marked STD. The participants conducted this evaluation in an odor neutral environment. The participants were notified the sample of the Prior Art composition (labeled STD) had an equivalent odor level of 10, and each participant was asked to rate samples A, B, C, and D on a basis of 1 to 10, with 10 representing an equivalent odor to the sample of the Prior Art composition and 1 being zero odor. The participants rated each sample A, B, C, and D compared to the Prior Art. The rate for each sample was averaged to determine the overall level of odor for the malodor neutralizing composition of each sample compared to the Prior Art composition. The averaged results for each sample compared to the Prior Art are found in Table E.

TABLE E

| Sample | Sensory Score |
|---|---|
| Prior Art | 10.0 |
| A | 7.1 |
| B | 6.1 |
| C | 5.6 |
| D | 4.0 |

In the results, the lower the number, the lower the intrinsic odor relative the intrinsic odor of the Prior Art composition. Based upon these test results, the participants determined the malodor neutralizing composition had a substantially reduced intrinsic odor compared to the prior art.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A malodor neutralizing composition comprising undecylenic acid or a derivative thereof, wherein the composition comprises a derivative of undecylenic acid of about 1 wt. % to about 99 wt. % methyl ester, about 1 wt. % to about 99 wt. % ethyl ester, and about 1 wt. % to about 99 wt. % heptyl ester, wherein the sum of the wt. % of the composition is 100%.

2. The malodor neutralizing composition according to claim 1, wherein the composition comprises about 10 wt. % to about 80 wt. % heptyl ester.

3. The malodor neutralizing composition according to claim 1, wherein the composition comprises from about 20 wt. % to about 80 wt. % methyl ester.

4. The malodor neutralizing composition according to claim 1, wherein the composition comprises from about 20 wt. % to about 80 wt. % ethyl ester.

5. The malodor neutralizing composition according to claim 1, wherein the composition comprises a ratio of ethyl ester to methyl ester from 5/95 to 30/70 that comprises from about 20 wt. % to about 90 wt. % of the composition and the heptyl ester comprises from about 10 wt. % to about 80 wt. % of the composition, wherein the sum of the wt. % of the composition is 100%.

6. The malodor neutralizing composition according to claim 1, wherein the composition comprises a ratio of ethyl ester to methyl ester of 80/20 that comprises about 70 wt. % of the composition and the heptyl ester comprises about 30 wt. % of the composition, wherein the sum of the wt. % of the composition is 100%.

7. A malodor neutralizing composition comprising undecylenic acid or a derivative thereof, wherein the composition comprises a derivative of undecylenic acid of about 1 wt. % to about 99 wt. % methyl ester; about 1 wt. % to about 99 wt. % ethyl ester, and about 10 wt. % to about 80 wt. % heptyl ester, wherein the sum of the wt. % of the composition is 100%.

8. The malodor neutralizing composition according to claim 7, wherein the composition comprises from about 20 wt. % to about 80 wt. % methyl ester.

9. The malodor neutralizing composition according to claim 7, wherein the composition comprises from about 20 wt. % to about 80 wt. % ethyl ester.

10. The malodor neutralizing composition according to claim 7, wherein the composition comprises a ratio of ethyl ester to methyl ester from 5/95 to 30/70 that comprises from about 20 wt. % to about 90 wt. % of the composition and the heptyl ester comprises from about 10 wt. % to about 80 wt. % of the composition, wherein the sum of the wt. % of the composition is 100%.

11. The malodor neutralizing composition according to claim 7, wherein the composition comprises a ratio of ethyl ester to methyl ester of 80/20 that comprises about 70 wt. % of the composition and the heptyl ester comprises about 30 wt. % of the composition, wherein the sum of the wt. % of the composition is 100%.

12. A composition for reducing malodors comprising:
a component selected from the group consisting of fragrances, flavors, and mixtures thereof;
an undecylenic acid or a derivative thereof, wherein the composition comprises a derivative of undecylenic acid of about 1 wt. % to about 99 wt. % methyl ester, about 1 wt. % to about 99 wt. % ethyl ester, and about 1 wt. % to about 99 wt. % heptyl ester, wherein the sum of the wt. % of the composition is 100%.

13. The malodor neutralizing composition according to claim 12, wherein the composition comprises about 10 wt. % to about 80 wt. % heptyl ester.

14. The malodor neutralizing composition according to claim 12, wherein the composition comprises from about 20 wt. % to about 80 wt. % methyl ester.

15. The malodor neutralizing composition according to claim 12, wherein the composition comprises from about 20 wt. % to about 80 wt. % ethyl ester.

16. The malodor neutralizing composition according to claim 12, wherein the composition comprises a ratio of ethyl ester to methyl ester from 5/95 to 30/70 that comprises from about 20 wt. % to about 90 wt. % of the composition and the heptyl ester comprises from about 10 wt. % to about 80 wt. % of the composition, wherein the sum of the wt. % of the composition is 100%.

17. The malodor neutralizing composition according to claim 12, wherein the composition comprises a ratio of ethyl ester to methyl ester of 80/20 that comprises about 70 wt. % of the composition and the heptyl ester comprises about 30 wt. % of the composition, wherein the sum of the wt. % of the composition is 100%.
